(19) United States
(12) Patent Application Publication
(10) Pub. No.: US 2021/0015458 A1
(43) Pub. Date: Jan. 21, 2021

(54) ULTRASOUND PROBE, METHOD OF MANUFACTURING ULTRASOUND PROBE, AND ULTRASOUND DIAGNOSTIC APPARATUS

(71) Applicant: Konica Minolta, Inc., Tokyo (JP)

(72) Inventor: Yuiko KITAMURA, Tokyo (JP)

(21) Appl. No.: 16/928,410

(22) Filed: Jul. 14, 2020

(30) Foreign Application Priority Data
Jul. 17, 2019 (JP) 2019-131890

(51) Int. Cl. A61B 8/00; B06B 1/06

(52) U.S. Cl. CPC A61B 8/4444; A61B 8/4483; B06B 1/0622

(57) ABSTRACT

An ultrasound probe includes: a piezoelectric material in which piezoelectric elements to transmit and receive ultrasound are one-dimensionally arrayed; and at least one acoustic matching layer arranged on a subject side of the piezoelectric material, wherein the piezoelectric material includes a plurality of first grooves, and at least a second groove formed between the plurality of first grooves, the piezoelectric material is divided by at least either the first grooves or the second groove, and either each of the first grooves or the second groove is a void, or the first grooves and the second groove are respectively filled with fillers having different hardness.

The entire disclosure of Japanese patent Application No. 2019-131890, filed on Jul. 17, 2019, is incorporated herein by reference in its entirety.

### BACKGROUND

### Technological Field

The present invention relates to an ultrasound probe, a method of manufacturing the ultrasound probe, and an ultrasound diagnostic apparatus including the ultrasound probe.

### Description of the Related Art

An ultrasound diagnostic apparatus has an ultrasound probe connected to the ultrasound diagnostic apparatus or capable of communicating with the ultrasound diagnostic apparatus, and the ultrasound diagnostic apparatus can obtain, as an ultrasound diagnostic image, a shape and movement of a tissue by placing the ultrasound probe on a body surface or inserting the ultrasound probe into a body of a subject including a human or another animal Since the ultrasound diagnostic apparatus is highly safe, there is a merit of being able to repeatedly perform examinations.

The ultrasound probe incorporates, for example, a piezoelectric element and the like that transmit and receive ultrasound. The piezoelectric element receives an electric signal (transmission signal) from the ultrasound diagnostic apparatus, converts the received transmission signal into an ultrasound signal, transmits the same, receives ultrasound reflected in a living body, converts the ultrasound into an electric signal (reception signal), and transmits the reception signal converted into the electric signal to the ultrasound diagnostic apparatus.

Furthermore, the ultrasound probe generally includes an acoustic matching layer on a living body side of the piezoelectric element, and acoustic impedance of the acoustic matching layer has magnitude between magnitude of acoustic impedance of the piezoelectric element and magnitude of acoustic impedance of the living body. The acoustic matching layer functions to match the acoustic impedance between the piezoelectric element and the subject (living body) and can increase resolution of an obtained ultrasound diagnostic image.

JP 63-164700 A discloses a method of manufacturing an ultrasound probe including: a step of performing first dicing at a required pitch in parallel to a width direction of a piezoelectric element so as to cut the piezoelectric element from one surface thereof to a position where the piezoelectric element is not completely separated, and then charging a filler to each of grooves formed by the dicing; and a step of forming a matching layer on the other surface of the piezoelectric element, performing second dicing in parallel to the width direction so as to cut the matching layer and the piezoelectric element from a surface side of the matching layer to a position continuous to the respective grooves of the piezoelectric element formed in the first dicing, and then charging the filler to the grooves formed in the second dicing. According to the above-described method of manufacturing the ultrasound probe, stable processing can be performed, and it is possible to provide the high-performance ultrasound probe and the method of manufacturing the same.

JP 9-238399 A discloses a method of manufacturing an ultrasound probe including: a step of cutting a piezoelectric transducer block at a required pitch to form a plurality of transducer elements; a step of fixing an integrated acoustic matching layer onto the piezoelectric transducer block; and a step of forming arrayed gaps in the acoustic matching layer in a manner conforming to the pitch of grooves cut between the transducer elements, in which a width of each arrayed gap is formed narrower than that of each gap between the arrayed transducer elements. According to the above-described method of manufacturing the ultrasound probe, it is possible to provide the ultrasound probe and the method of manufacturing the same in which an excellent ultrasound image having high diagnostic performance can be obtained by: forming the width of each cut gap between elements of the acoustic matching layer narrower than the width of each gap between the transducer elements; and filling the gaps between the transducer elements with a polymer resin having hardness lower than hardness of a material of the transducers.

JP 63-287200 A discloses a method of manufacturing an ultrasound probe including: a step of forming a primary base material by bonding a piezoelectric ceramic material layer and an acoustic matching layer onto a rear surface backing member; a step of cutting the primary base material at a predetermined pitch to form a secondary base material having a discontinuous cross section via gaps; and a step of filling the gaps with hollow particles having a fine average particle size. According to the above-described method of manufacturing the ultrasound probe, it is possible to provide an ultrasound probe and a method of manufacturing the same having excellent structural strength and excellent directivity in micro transducers.

According to study made by the present inventor, there is a problem in ultrasound probes obtained by all of methods of manufacturing the ultrasound probes disclosed in JP 63-164700 A, JP 9-238399 A, and JP 63-287200 A. The problem is that it is not possible to obtain an ultrasound probe having desired durability and desired acoustic characteristics because a filler and piezoelectric elements (a piezoelectric material and an acoustic matching layer) are delaminated from each other due to cure shrinkage of the filler charged into grooves that have been formed in the piezoelectric material and the acoustic matching layer. An additional problem is that a desired ultrasound probe cannot be stably manufactured due to such delamination between the filler and the piezoelectric elements (the piezoelectric material and the acoustic matching layer).

### SUMMARY

The present invention is made considering the above-described points and directed to providing an ultrasound probe, a method of manufacturing the ultrasound probe, and an ultrasound diagnostic apparatus including the ultrasound probe, in which a filler and piezoelectric elements (a piezoelectric material and an acoustic matching layer) are hardly delaminated from each other during manufacture.

To achieve the abovementioned object, according to an aspect of the present invention, an ultrasound probe reflecting one aspect of the present invention comprises: a piezoelectric material in which piezoelectric elements to transmit and receive ultrasound are one-dimensionally arrayed; and at least one acoustic matching layer arranged on a subject side of the piezoelectric material, wherein the piezoelectric material includes a plurality of first grooves, and at least a second groove formed between the plurality of first grooves, the piezoelectric material is divided by at least either the first grooves or the second groove, and either each of the first grooves or the second groove is a void, or the first grooves and the second groove are respectively filled with fillers having different hardness.

### BRIEF DESCRIPTION OF THE DRAWINGS

The advantages and features provided by one or more embodiments of the invention will become more fully understood from the detailed description given hereinbelow and the appended drawings which are given by way of illustration only, and thus are not intended as a definition of the limits of the present invention:

FIG. 1 is a cross-sectional view illustrating an exemplary entire structure of an ultrasound probe according to a first embodiment of the present invention;

FIG. 2 is a flowchart illustrating respective steps of a method of manufacturing the ultrasound probe according to the first embodiment of the present invention;

FIG. 3 is a cross-sectional view illustrating an exemplary entire structure of an ultrasound probe according to a second embodiment of the present invention;

FIG. 4 is a flowchart illustrating respective steps of a method of manufacturing the ultrasound probe according to the second embodiment of the present invention;

FIG. 5 is a cross-sectional view illustrating an exemplary entire structure of an ultrasound probe according to a third embodiment of the present invention;

FIG. 6 is a cross-sectional view illustrating an exemplary entire structure of an ultrasound probe according to a fourth embodiment of the present invention;

FIG. 7 is a flowchart illustrating respective steps of a method of manufacturing the ultrasound probe according to the fourth embodiment of the present invention;

FIG. 8 is a cross-sectional view illustrating an exemplary entire structure of an ultrasound probe according to a modified example of the present invention; and

FIG. 9 is a schematic diagram illustrating an exemplary ultrasound diagnostic apparatus including an ultrasound probe according to an embodiment of the present invention.

### DETAILED DESCRIPTION OF EMBODIMENTS

Hereinafter, one or more embodiments of the present invention will be described with reference to the drawings. However, the scope of the invention is not limited to the disclosed embodiments.

### First Embodiment

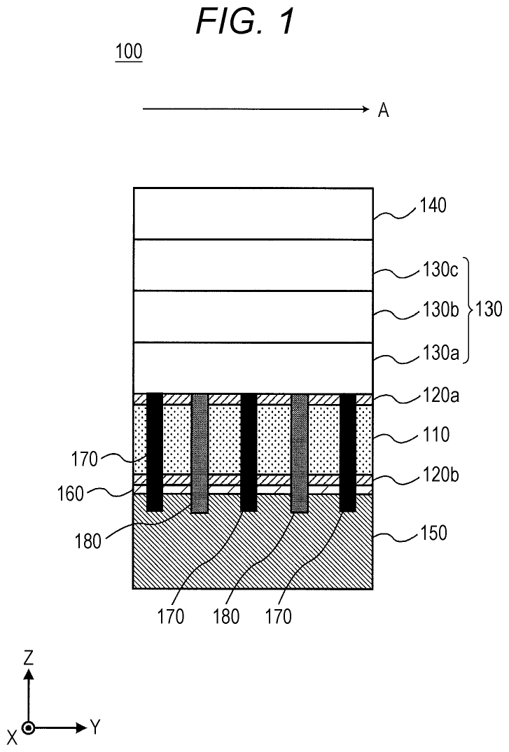

FIG. 1 is a cross-sectional view illustrating an exemplary entire structure of an ultrasound probe 100 according to a first embodiment of the present invention.

(Configuration of Ultrasound Probe)

As illustrated in FIG. 1, an ultrasound probe 100 according to the first embodiment includes a piezoelectric material 110, signal electrodes 120a and 120b provided to apply voltage to the piezoelectric material 110, at least one acoustic matching layer 130, an acoustic lens 140, a backing material 150, and a flexible printed circuit board (FPC) 160. The ultrasound probe 100 has a configuration in which the signal electrode 120a, the acoustic matching layer 130, and the acoustic lens 140 are laminated in this order from the piezoelectric material 110 toward a subject, and the signal electrode 120b, the flexible printed circuit board (FPC) 160, and the backing material 150 are laminated in this order from the piezoelectric material 110 toward an opposite side of the subject.

(Piezoelectric Material)

The piezoelectric material 110 transmits ultrasound by applying the voltage, and is formed by one-dimensionally arraying, in a Y direction in FIG. 1, a plurality of piezoelectric elements divided in an array direction (a direction A in FIG. 1) by grooves. The piezoelectric material 110 can have a thickness of, for example, 50 μm or more and 400 μm or less. The respective piezoelectric elements is formed from: for example, lead zirconate titanate (PZT)-based piezoelectric ceramics; piezoelectric single crystals of lead magnesium niobate/lead titanate solid solution (PMN-PT), lead zinc niobate/lead titanate solid solution (PZN-PT), and the like; a composite piezoelectric material obtained by combining these materials with a polymer material; and the like.

(Signal Electrodes)

The signal electrodes 120a and 120b are arranged on an upper surface side and a rear surface side of the piezoelectric material 110 respectively, and provided to apply the voltage to the piezoelectric material 110. The signal electrodes 120a and 120b can be formed by a method of performing deposition, sputtering, silver baking, or the like on gold, silver, and the like, or can be formed by pasting a conductor such as copper on an insulating substrate and then applying patterning thereto. Note that, in the present specification, a direction approaching a diagnosis subject will be referred to as an “upper surface side”, and a direction moving away from the diagnosis subject will be referred to as a “rear surface side” for the respective members constituting the ultrasound probe 100.

(Acoustic Matching Layer)

The acoustic matching layer 130 is a layer provided to match acoustic characteristics between the piezoelectric material 110 and the acoustic lens 140, and generally includes a plurality of layers. As illustrated in FIG. 1, in the first embodiment, the acoustic matching layer 130 includes a first acoustic matching layer 130a, a second acoustic matching layer 130b, and a third acoustic matching layer 130c.

(Acoustic Lens)

The acoustic lens 140 improves resolution by converging ultrasound transmitted from the piezoelectric material 110 by utilizing refraction caused by a sound speed difference between the subject (living body) and the acoustic lens 140. As illustrated in FIG. 1, in the first embodiment, the acoustic lens 140 is a cylindrical acoustic lens that extends in the Y direction in the drawing and has a convex shape in a Z direction. The acoustic lens 140 converges, in the Z direction, the ultrasound in the X direction, and emits the converged ultrasound to the outside of the ultrasound probe 100. Additionally, the acoustic lens 140 includes, for example, a soft polymer material such as silicone rubber having a sound speed different from that of the living body.

(Backing Material)

The backing material 150 is a layer that holds the piezoelectric material 110 and attenuates the ultrasound transmitted from the piezoelectric material 110 to a rear surface side thereof. The backing material 150 generally includes synthetic rubber, natural rubber, an epoxy resin, a thermoplastic resin, and the like obtained by charging a material to adjust acoustic impedance. The backing material 150 has a shape not particularly limited as far as the transmitted ultrasound can be attenuated.

(Flexible Printed Circuit Board)

The flexible printed circuit board (FPC) 160 is arranged in contact with the rear surface side of the signal electrode 120b, and connects the signal electrode 120b to an external power source and the like.

The piezoelectric material 110, the signal electrodes 120a and 120b, the respective layers of the acoustic matching layer 130, and the acoustic lens 140, the backing material 150, and the flexible printed circuit board (FPC) 160 may be bonded with an adhesive generally used in this technical filed, such as an epoxy based adhesive.

(Configuration of Piezoelectric Material)

Here, as illustrated in FIG. 1, the piezoelectric material 110 includes a plurality of first grooves 170 formed substantially in parallel, and second grooves 180 each formed between the plurality of first grooves 170 in a manner substantially in parallel to the first grooves 170, and the piezoelectric material 110 is divided by both the first grooves 170 and the second grooves 180. The number of the first grooves 170 and the number of the second grooves 180 formed in the piezoelectric material 110 may be the same or may be different.

Additionally, each of the first grooves 170 and each of the second grooves 180 formed in the piezoelectric material 110 have a width of 15 to 45 μm (in the direction A in FIG. 1). In a case of not dividing the piezoelectric material 110, a depth (in a downward direction in the drawing plane) of each groove is obtained by leaving an uncut portion equivalent to 10 to 20% relative to a thickness of the piezoelectric material 110. In a case of dividing the piezoelectric material 110, the depth of each groove is set to +10 to +100 μm relative to the thickness of the piezoelectric material. An interval at which the first grooves 170 are formed is 150 to 600 μm, and can be appropriately changed depending on the number of the second grooves 180 each formed between the first grooves 170.

Additionally, the width and the depth (the downward direction in the drawing plane) of each of the first grooves 170 and the second grooves 180 formed in the piezoelectric material 110 can be changed depending on a frequency (e.g., 2 to 20 MHz), and the interval at which the first grooves 170 and the second grooves 180 are formed can also be changed depending on the frequency. Note that the first grooves 170 and the second grooves 180 can be formed by using a dicing saw (manufactured by Disco Corporation).

Furthermore, either the first grooves 170 or the second grooves 180 formed in the piezoelectric material 110 are voids, or both kinds of the grooves are filled with fillers having different hardness. As the fillers, as far as the hardness is different, fillers including the same kind of the resin may be used. Note that the above-described fillers may be a mixed with a powdered aluminum oxide and the like.

Here, the fillers to be charged into each first groove 170 and each second groove 180 is preferably a resin selected from a group including an epoxy resin, a silicone resin, and a urethane resin.

Examples of the epoxy resin that can be used as the fillers include: epoxy resins of bisphenol types such as a bisphenol A type and a bisphenol F type; epoxy resins of novolak types including a resol novolak type and a phenol-modified novolak type; epoxy resins of polycyclic aromatic types such as a naphthalene structure-containing type, an anthracene structure-containing type, and a fluorene structure-containing type; an epoxy resin of an alicyclic type; and a liquid-crystalline epoxy resin. Examples of the silicone resin include RTV silicone rubber. Additionally, types of the silicone resin include a one-pack type, a two-pack type, a room temperature curable type, a heat curable type, a condensation reaction type, and an addition reaction type. Examples of the urethane resin include a thermosetting resin and a thermoplastic resin. Among the above-described resins, for example, a combination of an epoxy resin having Shore D hardness 80 with a silicone resin having Shore A hardness 35 is more preferable. Additionally, as the fillers, as far as the hardness is different, fillers including the same kind of the resin may be used. Here, the “Shore D hardness” and the “Shore A hardness” represent indentation hardness of rubber and elastomer measured in durometer hardness (JISK6253-3, Year 2012).

Additionally, as illustrated in FIG. 1, the ultrasound probe 100 may include the backing material 150 arranged on the rear surface side of the piezoelectric material 110. Furthermore, the backing material 150 may include at least either the first grooves 170 or the second grooves 180. In the first embodiment, the backing material 150 includes both the first grooves 170 and the second grooves 180.

Additionally, in the ultrasound probe 100 according to the first embodiment, the acoustic matching layer 130 is not divided. In this case, directivity of the ultrasound probe can be more improved by forming the acoustic matching layer 130 from a material having rubber elasticity, such as silicone rubber, chloroprene rubber, ethylene-propylene copolymer rubber, acrylonitrile-butadiene copolymer rubber, and urethane rubber. In this case, the above-described material having the rubber elasticity is preferably a material having a sound speed of 1650 msec or less.

(Effects)

In the ultrasound probe 100 according to the first embodiment, either the first grooves 170 or the second grooves 180 are formed as the voids or both kinds of the grooves are respectively filled with the fillers having the different hardness. As a result, cure shrinkage of the fillers can be absorbed, and delamination is hardly caused between the fillers and the piezoelectric material 110 during manufacture. In particular, since a filler having lower hardness is more easily deformed and tends to follow the cure shrinkage, when such a filler is used in combination with a filler having higher hardness, stress by the cure shrinkage is absorbed, and generation of delamination between the piezoelectric material 110 and the fillers can be suppressed.

Additionally, since the second grooves 180 are formed after filling each of the first grooves 170 with a first filler, stress caused by cure shrinkage of the first filler can be released. After that, when the second grooves 180 are filled with a second filler, cure shrinkage occurs in a manner similar to the case of the first filler. However, since the stress caused by the cure shrinkage of the first filler is absorbed, generation of delamination caused by the cure shrinkage can be more reduced compared to a case of forming all of the grooves at a time and filling all of the grooves with the fillers at a time. Furthermore, since all of the grooves are not filled with the fillers at a time, it is possible to reduce an amount of the fillers to be charged into the piezoelectric material 110 at a time. Therefore, influence of the cure shrinkage can be reduced.

Additionally, directivity of the ultrasound probe can be more improved by forming the acoustic matching layer from the material having the rubber elasticity, such as the silicone rubber, the chloroprene rubber, the ethylene-propylene copolymer rubber, the acrylonitrile-butadiene copolymer rubber, and the urethane rubber.

Consequently, it is possible to obtain an ultrasound probe having desired durability, desired acoustic characteristics, and excellent productivity.

(Method of Manufacturing Ultrasound Probe)

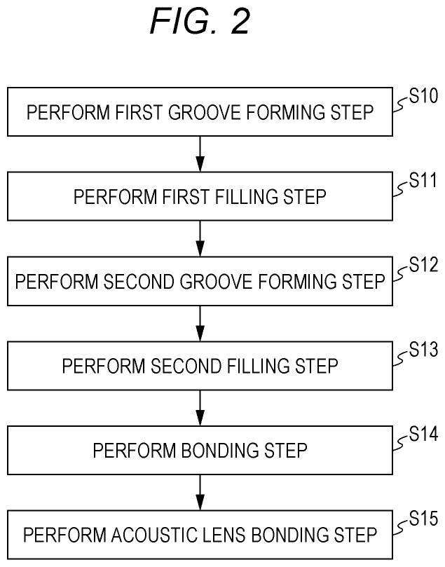

A method of manufacturing the ultrasound probe 100 according to the first embodiment will be described using a flowchart illustrated in FIG. 2.

The method of manufacturing the ultrasound probe 100 according to the first embodiment includes: a first groove forming step (S10) of forming a plurality of first grooves 170 in the piezoelectric material 110; a first filling step (S11) of filling each of the first grooves 170 with the first filler; a second groove forming step (S12) of forming each of second grooves 180 between the first grooves 170 in the piezoelectric material 110; a second filling step (S13) of filling each of the second grooves 180 with the second filler; a bonding step (S14) of bonding the acoustic matching layer 130 arranged on the subject side of the piezoelectric material 110; and an acoustic lens bonding step (S15) of bonding the acoustic lens 140 to an uppermost layer (the third acoustic matching layer 130c) of the acoustic matching layer 130. In the present invention, the “uppermost layer” refers to an acoustic matching layer arranged at a position closest to the subject on the above-described upper surface side. In the present embodiment, the uppermost layer refers to the third acoustic matching layer 130c.

Note that, in the present invention, “to bond” means to bond the acoustic matching layer and the like by using, for example, an epoxy-based or silicone-based thermosetting adhesive.

Here, both the first groove forming step (S10) and the second groove forming step (S12) include steps of forming the grooves that divide the piezoelectric material 110. In the piezoelectric material 110, the thickness, the groove width, the groove depth, and the interval at which the grooves are formed are changed depending on the frequency. For example, in a case where: the frequency is 7.5 MHz; the piezoelectric element pitch is 200 μm; and the number of piezoelectric elements is 192, the first grooves 170 are formed substantially in parallel at the interval of 200 μm in the first groove forming step (S10) while each groove has the width of 20 to 30 μm and the depth of +10 to 100 μm relative to the thickness of the piezoelectric material 110. In the second groove forming step (S12), each second groove 180 having the width of 20 to 30 μm is formed between the first grooves 170. Each second groove 180 formed in the second groove forming step (S12) is formed at a position 100 μm away from each first groove 170.

The first groove forming step (S10) and the second groove forming step (S12) can be performed by utilizing a known processing method of a piezoelectric material. In general, the first grooves 170 and the second grooves 180 can be formed by using a dicing saw. Additionally, in a case where the piezoelectric material 110 has a thickness of 10 μm or more, the grooves can be formed by a known processing machine such as a diamond cutter. In a case where the piezoelectric material 110 has a thickness of less than 10 μm, the grooves can be formed by micro electro mechanical systems (MEMS) processing.

Furthermore, in the method of manufacturing the ultrasound probe 100 according to the first embodiment, a step of bonding the backing material 150 to the rear surface side of the piezoelectric material 110 (the step not illustrated) may be included before the first groove forming step (S10) in addition to the steps illustrated in the flowchart of FIG. 2. Here, in the ultrasound probe 100 having the backing material 150 bonded to the rear surface side of the piezoelectric material 110, either the first groove forming step (S10) of forming the plurality of first grooves 170 in the piezoelectric material 110 or the second groove forming step (S12) of forming each of the second grooves 180 between the first grooves 170 in the piezoelectric material 110 may include a step of forming the first grooves 170 or the second grooves 180 in the backing material 150.

Additionally, as for the fillers to be charged into each of the first grooves 170 formed in the first groove forming step (S10) and each of the second grooves 180 formed in the second groove forming step (S12), the filler to be charged into either the first groove 170 or the second groove 180 is the air, or the respective fillers are the fillers having the different hardness. The first filler to be charged into the first grooves 170 is preferably a resin selected from the group including the epoxy resin, the silicone resin, and the urethane resin. The second filler to be charged into the second grooves 180 is preferably a resin having the hardness different from that of the first filler and is selected from the group including the epoxy resin, the silicone resin, and the urethane resin.

Examples of the epoxy resin that can be used as the fillers include: the epoxy resins of bisphenol types such as the bisphenol A type and the bisphenol F type; the epoxy resins of the novolak types including the resol novolak type and the phenol-modified novolak type; the epoxy resins of the polycyclic aromatic types such as the naphthalene structure-containing type, the anthracene structure-containing type, and the fluorene structure-containing type; the epoxy resin of the alicyclic type; and the liquid-crystalline epoxy resin. Examples of the silicone resin include the RTV silicone rubber. Additionally, the types of the silicone resin include the one-pack type, the two-pack type, the room temperature curable type, the heat curable type, the condensation reaction type, and the addition reaction type. Examples of the urethane resin include the thermosetting resin and the thermoplastic resin. Among the above-described resins, for example, the combination of the epoxy resin having the Shore D hardness 80 with the silicone resin having the Shore A hardness 35 is more preferable. Additionally, as the fillers, as far as the hardness is different, the fillers including the same kind of the resin may be used.

(Effects)

In the method of manufacturing the ultrasound probe 100 according to the first embodiment, either the first grooves 170 or the second grooves 180 are formed as the voids or both kinds of the grooves are respectively filled with the fillers having the different hardness. As a result, cure shrinkage of the fillers can be absorbed. Therefore, it is possible to manufacture an ultrasound probe in which delamination between the fillers and the respective grooves formed in the piezoelectric material 110 is suppressed. In particular, since the filler having lower hardness is more easily deformed and tends to follow the cure shrinkage, when such a filler is used in combination with a filler having higher hardness, stress by the cure shrinkage is absorbed, and generation of delamination between the fillers and the piezoelectric material 110 can be suppressed.

Additionally, since the second groove forming step (S12) is performed after the first filling step (S11), it is possible to release the stress caused by the cure shrinkage of the first filler. Here, when the second filler is charged, cure shrinkage occurs in a manner similar to the case of the first filler. However, since the stress caused by the cure shrinkage of the first filler is absorbed, generation of delamination caused by the cure shrinkage can be more reduced compared to a case of forming all of the grooves at a time and filling all of the grooves with the fillers at a time. Furthermore, since all of the grooves are not filled with the fillers at a time, it is possible to reduce an amount of the fillers to be charged into the piezoelectric material 110 at a time. Therefore, influence of the cure shrinkage can be reduced.

Moreover, since the first groove forming step (S10) and the first filling step (S11) are performed separately from the second groove forming step (S12) and the second filling step (S13), a desired groove can be surely filled with a desired filler.

Consequently, it is possible to obtain the ultrasound probe having the desired durability, the desired acoustic characteristics, and the excellent productivity.

### Second Embodiment

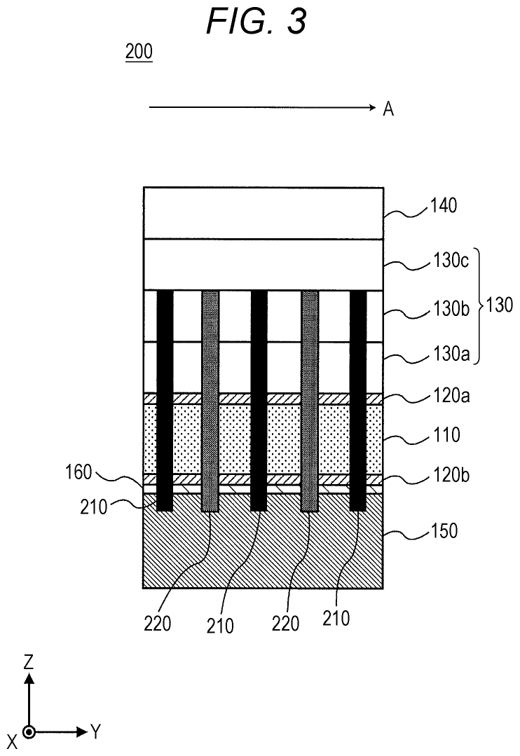

FIG. 3 is a cross-sectional view illustrating an exemplary entire structure of an ultrasound probe 200 according to a second embodiment of the present invention.

(Configuration of Ultrasound Probe)

The ultrasound probe 200 according to the second embodiment differs from an ultrasound probe 100 according to a first embodiment only in that an acoustic matching layer 130 includes layers (hereinafter, also referred to as “division layers”) divided by both first grooves 210 and second grooves 220. Accordingly, components same as those of the ultrasound probe 100 according to the first embodiment will be denoted by the same reference signs, and a description thereof will be omitted.

As illustrated in FIG. 3, in the ultrasound probe 200 according to the second embodiment, the acoustic matching layer 130 includes: the division layers (a first acoustic matching layer 130a and a second acoustic matching layer 130b) divided by at least either the first grooves 210 or the second grooves 220; and a non-division layer (a third acoustic matching layer 130c) not divided by any one of the first grooves 210 and the second grooves 220.

In the ultrasound probe 200 illustrated in FIG. 3, the first acoustic matching layer 130a, the second acoustic matching layer 130b, and the piezoelectric material 110 are divided by both the first grooves 210 and the second grooves 220, but the present invention is not limited thereto. Only the first acoustic matching layer 130a and the piezoelectric material 110 may be divided by the first grooves 210 and the second grooves 220, or all of the acoustic matching layers including the third acoustic matching layer 130c may be divided by the first grooves 210 and the second grooves 220.

Additionally, the division layers may be layers divided by both the first grooves 210 and the second grooves 220, or may be layers divided by only either the first grooves 210 or the second grooves 220.

Additionally, either the first grooves 210 or the second grooves 220 are voids, or both kinds of the grooves are filled with fillers respectively having different hardness. As the above-described fillers, as far as the hardness is different, fillers including the same kind of the resin may be used. Here, the fillers to be charged into the first grooves 210 and the second grooves 220 include preferably a resin selected from a group including an epoxy resin, a silicone resin, and a urethane resin described above. For example, a combination of an epoxy resin having Shore D hardness 80 with a silicone resin having Shore A hardness 35 is more preferable.

Additionally, as illustrated in FIG. 3, the ultrasound probe 200 may include a backing material 150 arranged on a rear surface side of the piezoelectric material 110. Furthermore, the backing material 150 may include at least either the first grooves 210 or the second grooves 220. In the second embodiment, the backing material 150 includes both the first grooves 210 and the second grooves 220.

(Effects)

In the ultrasound probe 200 according to the second embodiment, either the first grooves 210 or the second grooves 220 are formed as the voids or both kinds of the grooves are respectively filled with the fillers having the different hardness. As a result, cure shrinkage of the fillers can be absorbed, and it is possible to hardly cause delamination between the fillers and the piezoelectric elements (piezoelectric material 110 and the acoustic matching layer 130) during manufacture. In particular, since a filler having lower hardness is more easily deformed and tends to follow the cure shrinkage, when such a filler is used in combination with a filler having higher hardness, stress by the cure shrinkage is absorbed, and generation of delamination between the fillers and the piezoelectric elements (the piezoelectric material 110 and the acoustic matching layer 130) can be suppressed.

Additionally, since the second grooves 220 are formed after filling each of the first grooves 210 with a first filler, stress caused by cure shrinkage of the first filler can be released. After that, when the second grooves 220 are filled with a second filler, cure shrinkage occurs in a manner similar to the case of the first filler. However, since the stress caused by the cure shrinkage of the first filler is absorbed, generation of delamination caused by the cure shrinkage can be more reduced compared to a case of forming all of the grooves at a time and filling all of the grooves with the fillers at a time. Furthermore, since all of the grooves are not filled with the fillers at a time, it is possible to reduce an amount of the fillers to be charged into the piezoelectric material 110 and the inside of the acoustic matching layer 130 at a time. Therefore, influence of the cure shrinkage can be reduced.

Consequently, it is possible to obtain an ultrasound probe having desired durability, desired acoustic characteristics, and excellent productivity.

(Method of Manufacturing Ultrasound Probe)

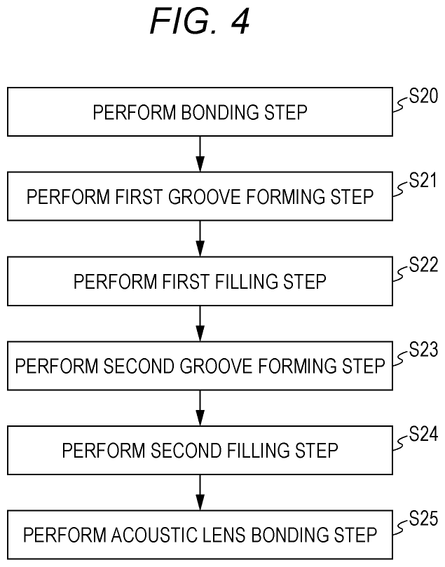

A method of manufacturing the ultrasound probe 200 according to the second embodiment will be described using a flowchart illustrated in FIG. 4.

The method of manufacturing the ultrasound probe 200 according to the second embodiment includes: a bonding step (S20) of bonding the acoustic matching layer 130 arranged on a subject side of the piezoelectric material 110; a first groove forming step (S21) of forming the plurality of first grooves 210 in the piezoelectric material 110 and the acoustic matching layer 130; a first filling step (S22) of filling each of the first grooves 210 with the first filler; a second groove forming step (S23) of forming each of the second grooves 220 between the plurality of first grooves 210 in the piezoelectric material 110 and the acoustic matching layer 130; a second filling step (S24) of filling each of the second grooves 220 with the second filler; and an acoustic lens bonding step (S25) of bonding an acoustic lens 140 to an uppermost layer (the third acoustic matching layer 130c) of the acoustic matching layer 130. Here, at least one of the first groove forming step (S21) and the second groove forming step (S23) includes a step of forming grooves that divide the piezoelectric material 110.

Note that a bonding step of bonding the acoustic matching layer may be further provided after the second filling step (S24). Consequently, it is possible to manufacture an ultrasound probe including the acoustic matching layer that includes: the division layers (the first acoustic matching layer 130a and the second acoustic matching layer 130b) divided by both the first grooves 210 and the second grooves 220; and the non-division layer (third acoustic matching layer 130c) not divided by any one of the first grooves 210 and the second grooves 220.

Additionally, as for the fillers to be charged into the first grooves 210 formed in the first groove forming step (S21) and the second grooves 220 formed in the second groove forming step (S23), the filler to be charged into either the first grooves 210 or the second groove 220 is the air, or the fillers to be charged into both kinds of grooves are the fillers having the different hardness. The first filler to be charged into the formed first grooves 210 is preferably a resin selected from the group including the epoxy resin, the silicone resin, and the urethane resin. The second filler to be charged into the formed second groove 220 is preferably a resin having the hardness different from that of the first filler and is selected from the group including the epoxy resin, the silicone resin, and the urethane resin. For example, the combination of the epoxy resin having the Shore D hardness 80 with the silicone resin having the Shore A hardness 35 is more preferable. Additionally, as the fillers, as far as the hardness is different, fillers including the same kind of the resin may be used.

Furthermore, in the method of manufacturing the ultrasound probe 200 according to the second embodiment, a step of bonding the backing material 150 to the rear surface side of the piezoelectric material 110 (the step not illustrated) may be included before the bonding step (S20) in addition to the steps illustrated in the flowchart of FIG. 4. Here, in the ultrasound probe 200 having the backing material 150 bonded to the rear surface side of the piezoelectric material 110, either the first groove forming step (S21) of forming the plurality of first grooves 210 in the piezoelectric material 110 or the second groove forming step (S23) of forming each of the second grooves 220 between the first grooves 210 in the piezoelectric material 110 may include a step of forming the first grooves 210 or the second grooves 220 in the backing material 150.

(Effects)

In the method of manufacturing the ultrasound probe 200 according to the second embodiment, either the first grooves 210 or the second grooves 220 are formed as the voids, or both kinds of the grooves are respectively filled with the fillers having the different hardness. As a result, even when the respective grooves have the same depths, cure shrinkage of the fillers can be absorbed. Therefore, it is possible to manufacture an ultrasound probe in which delamination between the fillers and the respective grooves formed in the piezoelectric elements (piezoelectric material 110 and the acoustic matching layer 130) is suppressed. In particular, since the filler having lower hardness is more easily deformed and tends to follow the cure shrinkage, when such a filler is used in combination with a filler having higher hardness, stress by the cure shrinkage is absorbed, and generation of delamination between the fillers and the piezoelectric elements (the piezoelectric material 110 and the acoustic matching layer 130) can be suppressed.

Additionally, since the second groove forming step (S23) is performed after the first filling step (S22), it is possible to release the stress caused by the cure shrinkage of the first filler. After that, when the second filler is charged, cure shrinkage occurs in a manner similar to the case of the first filler. However, since the stress caused by the cure shrinkage of the first filler is absorbed, generation of delamination caused by the cure shrinkage can be more reduced compared to a case of forming all of the grooves at a time and filling all of the grooves with the fillers at a time. Furthermore, since all of the grooves are not filled with the fillers at a time, it is possible to reduce an amount of the fillers to be charged into the piezoelectric elements (piezoelectric material 110 and the acoustic matching layer 130) at a time. Therefore, influence of the cure shrinkage can be reduced.

Moreover, since the first groove forming step (S21) and the first filling step (S22) are performed separately from the second groove forming step (S23) and the second filling step (S24), a desired groove can be filled with a desired filler.

Consequently, it is possible to obtain the ultrasound probe having the desired durability, the desired acoustic characteristics, and the excellent productivity.

### Third Embodiment

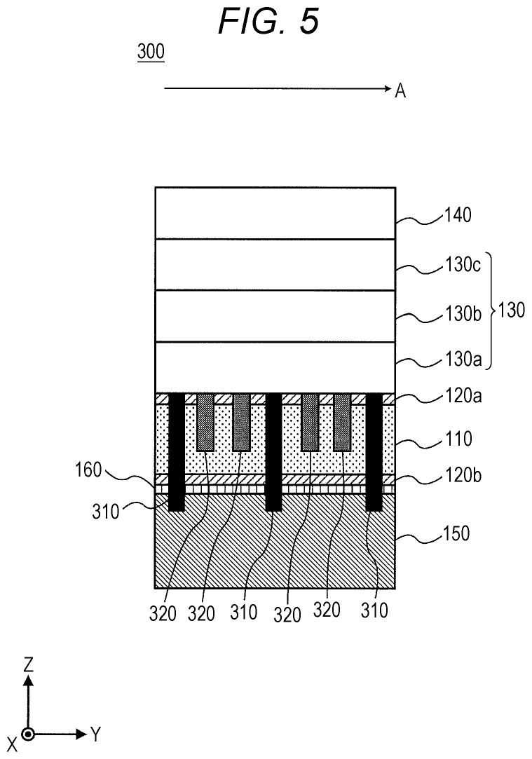

FIG. 5 is a cross-sectional view illustrating an exemplary entire structure of an ultrasound probe 300 according to a third embodiment of the present invention.

(Configuration of Ultrasound Probe)

The ultrasound probe 300 according to the third embodiment differs from an ultrasound probe 100 according to a first embodiment only in that only either first grooves 310 or second grooves 320 divide a piezoelectric material 110. Accordingly, components same as those of the ultrasound probe 100 according to the first embodiment will be denoted by the same reference signs, and a description thereof will be omitted.

As illustrated in FIG. 5, in the ultrasound probe 300 according to the third embodiment, the piezoelectric material 110 is divided only by the first grooves 310, but the present invention is not limited thereto. In the third embodiment, the piezoelectric material 110 may be divided by only the second grooves 320. Additionally, as illustrated in FIG. 5, two second grooves 320 are formed between two first grooves 310 in the ultrasound probe 300 according to the third embodiment, but not limited thereto, one second groove 320 may be formed between two first grooves 310 instead, or three or more second grooves 320 may be formed between two first grooves 310.

Furthermore, in FIG. 5, an acoustic matching layer 130 is not divided by any one of the first grooves 310 and the second grooves 320, but the present invention is not limited thereto. For example, some layers such as a first acoustic matching layer 130a may be divided by both the first grooves 310 and the second grooves 320 or either one thereof, or all of layers of the acoustic matching layer 130 including an uppermost layer (a third acoustic matching layer 130c) may be divided by both the first grooves 310 and the second grooves 320 or either one thereof.

Furthermore, either each first groove 310 or each second groove 320 that does not divide the piezoelectric material 110 has a depth not particularly limited as far as the depth is not enough to divide the piezoelectric material 110. The depth of the first groove 310 or the second groove 320 in the case of not dividing the piezoelectric material 110 is preferably 80 to 90% of a height of the piezoelectric material 110.

Additionally, either the first grooves 310 or the second grooves 320 constituting the division layer are voids or both kinds of the grooves are respectively filled with fillers having different hardness. As the above-described fillers, as far as the hardness is different, fillers including the same kind of the resin may be used. Here, the fillers to be charged into the first grooves 310 and the second grooves 320 include preferably a resin selected from a group including an epoxy resin, a silicone resin, and a urethane resin described above. For example, a combination of an epoxy resin having Shore D hardness 80 with a silicone resin having Shore A hardness 35 is more preferable.

Additionally, as illustrated in FIG. 5, the ultrasound probe 300 may include a backing material 150 arranged on a rear surface side of the piezoelectric material 110. Furthermore, the backing material 150 may include at least either the first grooves 310 or the second grooves 320. In the third embodiment, the backing material 150 includes the first grooves 310.

(Effects)

In the ultrasound probe 300 according to the third embodiment, either the first grooves 310 or the second grooves 320 are formed as the voids or both kinds of the grooves are respectively filled with the fillers having the different hardness. As a result, cure shrinkage of the fillers can be absorbed, and it is possible to hardly cause delamination between the fillers and the piezoelectric material during manufacture. In particular, since a filler having lower hardness is more easily deformed and tends to follow cure shrinkage, when such a filler is used in combination with a filler having higher hardness, stress by the cure shrinkage is absorbed, and generation of delamination between the fillers and the piezoelectric material 110 can be suppressed. Additionally, since the depth of each first groove 310 to be formed is made different from the depth of each second groove 320 to be formed, an amount of the filler to be charged into shallower grooves can be reduced. Therefore, the cure shrinkage of the fillers can be reduced.

Additionally, since the second grooves 320 are formed by filling a second filler after filling each of the first grooves 310 with a first filler, stress caused by cure shrinkage of the first filler can be released. Here, when the second filler is charged, cure shrinkage occurs in a manner similar to the case of the first filler. However, since the stress caused by the cure shrinkage of the first filler is absorbed, generation of delamination caused by the cure shrinkage can be more reduced compared to a case of forming all of the grooves at a time and filling all of the grooves with the fillers at a time. Furthermore, since all of the grooves are not filled with the fillers at a time, it is possible to reduce an amount of the fillers to be charged into the piezoelectric material 110 at a time. Therefore, influence of the cure shrinkage can be reduced.

Additionally, directivity of the ultrasound probe can be more improved by forming the acoustic matching layer from a material having rubber elasticity, such as silicone rubber, chloroprene rubber, ethylene-propylene copolymer rubber, acrylonitrile-butadiene copolymer rubber, and urethane rubber.

Consequently, it is possible to obtain an ultrasound probe having desired durability, desired acoustic characteristics, and excellent productivity.

(Method of Manufacturing Ultrasound Probe)

The method of manufacturing the ultrasound probe 300 according to the third embodiment can be manufactured in a manner similar to a flowchart illustrated in FIG. 2. Steps same as those in the first embodiment are denoted by the same reference signs, and a description thereof will be omitted.

The method of manufacturing the ultrasound probe 300 according to the third embodiment includes: a first groove forming step (S10) of forming the plurality of first grooves 310 in the piezoelectric material 110; a first filling step (S11) of filling each of the first grooves 310 with the first filler; a second groove forming step (S12) of forming the second grooves 320 between the first grooves 310 in the piezoelectric material 110; a second filling step (S13) of filling each of the second grooves 320 with the second filler; a bonding step (S14) of bonding the acoustic matching layer 130 arranged on a subject side of the piezoelectric material 110; and a step (S15) of bonding an acoustic lens 140 to an uppermost layer (the third acoustic matching layer 130c) of the acoustic matching layer 130. Here, at least one of the first groove forming step (S10) and the second groove forming step (S13) includes a step of forming the grooves that divide the piezoelectric material 110, and the other one of the steps includes a step of forming grooves that do not divide the piezoelectric material 110.

Additionally, either the first grooves 310 or the second grooves 320 constituting the division layer are the voids or both kinds of the grooves are respectively filled with the fillers having different hardness. As the above-described fillers, as far as the hardness is different, fillers including the same kind of the resin may be used. Here, the fillers to be charged into the first grooves 310 and the second grooves 320 include preferably the resin selected from the group including the epoxy resin, the silicone resin, and the urethane resin described above. For example, the combination of the epoxy resin having the Shore D hardness 80 with the silicone resin having the Shore A hardness 35 is more preferable. Additionally, as the fillers, as far as the hardness is different, fillers including the same kind of the resin may be used.

Furthermore, in the method of manufacturing the ultrasound probe 300 according to the third embodiment, a step of bonding the backing material 150 to the rear surface side of the piezoelectric material 110 (the step not illustrated) may be included before the first groove forming step (S10) in addition to the steps illustrated in the flowchart of FIG. 2. Here, in the ultrasound probe 300 having the backing material 150 bonded to the rear surface side of the piezoelectric material 110, either the first groove forming step (S10) of forming the plurality of first grooves 310 in the piezoelectric material 110 or the second groove forming step (S12) of forming the second groove(s) 320 between the first grooves 310 in the piezoelectric material 110 may include a step of forming the first grooves 310 or the second grooves 320 in the backing material 150.

(Effects)

In the method of manufacturing the ultrasound probe 300 according to the third embodiment, either the first grooves 310 or the second grooves 320 are formed as the voids, or both kinds of the grooves are respectively filled with the fillers having the different hardness. As a result, cure shrinkage of the fillers can be absorbed. Therefore, it is possible to manufacture an ultrasound probe in which delamination between the fillers and the respective grooves formed in the piezoelectric material 110 is suppressed. In particular, since the filler having lower hardness is more easily deformed and tends to follow the cure shrinkage, when such a filler is used in combination with a filler having higher hardness, stress by the cure shrinkage is absorbed, and generation of delamination between the fillers and the piezoelectric material 110 can be suppressed. Furthermore, since the depth of each first groove 310 to be formed is made different from the depth of each second groove 320 to be formed, the amount of the filler to be charged into the shallower groove can be reduced. Therefore, the cure shrinkage of the fillers can be reduced.

Additionally, since the second groove forming step (S12) is performed after the first filling step (S11), it is possible to release the stress caused by the cure shrinkage of the first filler. After that, when the second filler is charged, cure shrinkage occurs in a manner similar to the case of the first filler. However, since the stress caused by the cure shrinkage of the first filler is absorbed, generation of delamination caused by the cure shrinkage can be more reduced compared to a case of forming all of the grooves at a time and filling all of the grooves with the fillers at a time. Furthermore, since all of the grooves are not filled with the fillers at a time, it is possible to reduce an amount of the fillers to be charged into the piezoelectric material 110 at a time. Therefore, influence of the cure shrinkage can be reduced.

Moreover, since the first groove forming step (S10) and the first filling step (S11) are performed separately from the second groove forming step (S12) and the second filling step (S13), a desired groove can be filled with a desired filler.

Consequently, it is possible to obtain the ultrasound probe having the desired durability, the desired acoustic characteristics, and the excellent productivity.

### Fourth Embodiment

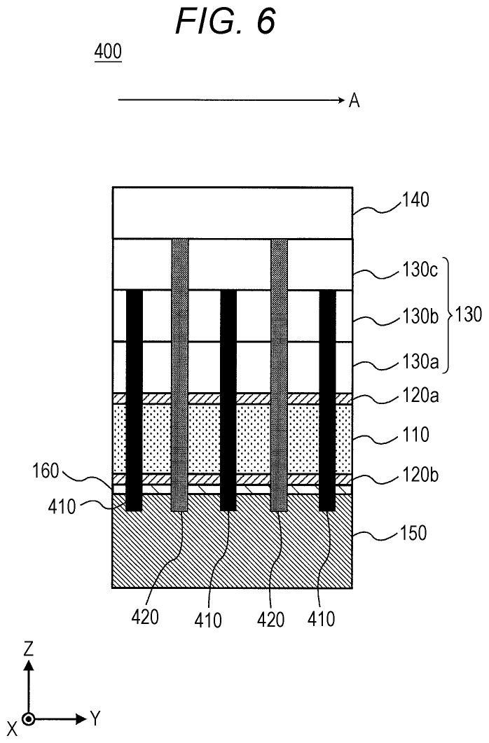

FIG. 6 is a cross-sectional view illustrating an exemplary entire structure of an ultrasound probe 400 according to a fourth embodiment of the present invention.

(Configuration of Ultrasound Probe)

The ultrasound probe 400 according to the fourth embodiment differs from an ultrasound probe 100 of a first embodiment only in that an acoustic matching layer 130 includes: layers divided by both first grooves 410 and second grooves 420; and an uppermost layer divided by either the first grooves 410 or the second grooves 420. Accordingly, components same as those of the ultrasound probe 100 according to the first embodiment will be denoted by the same reference signs, and a description thereof will be omitted.

As illustrated in FIG. 6, in the ultrasound probe 400 according to the fourth embodiment, a first acoustic matching layer 130a and a second acoustic matching layer 130b are divided by the first grooves 410, and all of the acoustic matching layers including the uppermost layer (a third acoustic matching layer 130c) are divided by the second grooves 420. Note that only either the first acoustic matching layer 130a or the second acoustic matching layer 130b may be divided by the first grooves 410.

In FIG. 6, the uppermost layer (the third acoustic matching layer 130c) is divided only by the second grooves 420, but the present embodiment is not limited thereto. In the present embodiment, the uppermost layer (the third acoustic matching layer 130c) may be divided only by the first grooves 410. Additionally, in FIG. 6, a piezoelectric material 110 is divided by both the first grooves 410 and the second grooves 420, but the piezoelectric material 110 may be divided by either the first grooves 410 or the second grooves 420.

Additionally, either the first grooves 410 or the second grooves 420 are voids or both kinds of the grooves are respectively filled with fillers having different hardness. As the above-described fillers, as far as the hardness is different, fillers including the same kind of the resin may be used. Here, the fillers to be charged into the first grooves 410 and the second grooves 420 include preferably a resin selected from a group including an epoxy resin, a silicone resin, and a urethane resin described above. For example, a combination of an epoxy resin having Shore D hardness 80 with a silicone resin having Shore A hardness 35 is more preferable.

Additionally, as illustrated in FIG. 6, the ultrasound probe 400 may include a backing material 150 arranged on a rear surface side of the piezoelectric material 110. Furthermore, the backing material 150 may include at least either the first grooves 410 or the second grooves 420. In the fourth embodiment, the backing material 150 includes both the first grooves 410 and the second grooves 420.

(Effects)

In the ultrasound probe 400 according to the fourth embodiment, either the first grooves 410 or the second grooves 420 are formed as the voids or both kinds of the grooves are respectively filled with the fillers having the different hardness. As a result, cure shrinkage of the fillers can be absorbed, and it is possible to hardly cause delamination between the fillers and the piezoelectric elements (piezoelectric material 110 and the acoustic matching layer 130) during manufacture. In particular, since a filler having lower hardness is more easily deformed and tends to follow the cure shrinkage, when such a filler is used in combination with a filler having higher hardness, stress by the cure shrinkage is absorbed, and generation of delamination between the fillers and the piezoelectric elements (the piezoelectric material 110 and the acoustic matching layer 130) can be suppressed. Furthermore, since only either the first grooves 410 or the second grooves 420 divide the uppermost layer (the third acoustic matching layer 130c) of the acoustic matching layer 130, it is possible to reduce an amount of the filler to be charged into the uppermost layer (the third acoustic matching layer 130c) of the acoustic matching layer 130 that is likely to be a start point of delamination. Therefore, delamination caused by the cure shrinkage can be reduced.

Additionally, since the second grooves are formed after the first filler is charged, the stress caused by the cure shrinkage of the first filler can be released. After that, when the second filler is charged, cure shrinkage occurs in a manner similar to the case of the first filler. However, since the stress caused by the cure shrinkage of the first filler is absorbed, generation of delamination caused by the cure shrinkage can be more reduced compared to a case of forming all of the grooves at a time and filling all of the grooves with the fillers at a time. Furthermore, since all of the grooves are not filled with the fillers at a time, it is possible to reduce an amount of the fillers to be charged into the piezoelectric elements (piezoelectric material 110 and the acoustic matching layer 130) at a time. Therefore, influence of the cure shrinkage can be reduced.

Consequently, it is possible to obtain an ultrasound probe having desired durability, desired acoustic characteristics, and excellent productivity.

(Method of Manufacturing Ultrasound Probe)

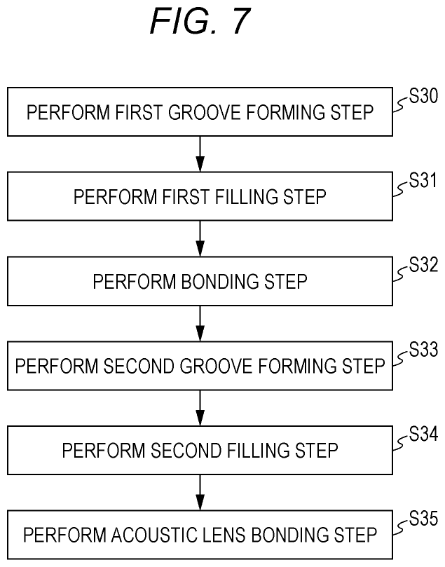

A method of manufacturing the ultrasound probe 400 according to the fourth embodiment will be described using a flowchart illustrated in FIG. 7.

The method of manufacturing the ultrasound probe 400 according to the fourth embodiment sequentially includes: a first groove forming step (S30) of forming the plurality of first grooves 410 in the piezoelectric material 110 and the acoustic matching layers 130a and 130b; a first filling step (S31) of filling each of the first grooves 410 with the first filler; a bonding step (S32) of bonding the third acoustic matching layer 130c arranged on a subject side of the piezoelectric material 110 (also a division layer bonding step of bonding a division layer to be divided by the second grooves 420); a second groove forming step (S33) of forming each of the second grooves 420 between the plurality of first grooves 410 in the piezoelectric material 110 having the acoustic matching layer 130 bonded; a second filling step (S34) of filling each of the second grooves 420 with the second filler; and an acoustic lens bonding step (S35) of bonding an acoustic lens 140 to the uppermost layer (the third acoustic matching layer 130c) of the acoustic matching layer 130.

Additionally, either the first grooves 410 or the second grooves 420 constituting the division layer are the voids or both kinds of the grooves are respectively filled with the fillers having the different hardness. As the above-described fillers, as far as the hardness is different, fillers including the same kind of the resin may be used. Here, the fillers to be charged into the first grooves 410 and the second grooves 420 include preferably a resin selected from the group including the epoxy resin, the silicone resin, and the urethane resin described above. For example, the combination of the epoxy resin having the Shore D hardness 80 with the silicone resin having the Shore A hardness 35 is more preferable. Additionally, as the fillers, as far as the hardness is different, fillers including the same kind of the resin may be used.

Furthermore, in the method of manufacturing the ultrasound probe 400 according to the fourth embodiment, a step of bonding a backing material 150 to the rear surface side of the piezoelectric material 110 (the step not illustrated) may be included before the first groove forming step (S30) in addition to the steps illustrated in the flowchart of FIG. 7. Here, in the ultrasound probe 400 having the backing material 150 bonded to the rear surface side of the piezoelectric material 110, either the first groove forming step (S30) of forming the plurality of first grooves 410 in the piezoelectric material 110 or the second groove forming step (S33) of forming each of the second grooves 420 between the first grooves 410 in the piezoelectric material 110 may include a step of forming the first grooves 410 or the second grooves 420 in the backing material 150.

(Effects)

In the method of manufacturing the ultrasound probe 400 according to the fourth embodiment, either the first grooves 410 or the second grooves 420 are formed as the voids or both kinds of the grooves are respectively filled with the fillers having the different hardness. As a result, it is possible to manufacture an ultrasound probe in which delamination between the fillers and the respective grooves formed in the piezoelectric elements (the piezoelectric material 110 and the acoustic matching layer 130) is suppressed. In particular, since a filler having lower hardness is more easily deformed and tends to follow the cure shrinkage, when such a filler is used in combination with a filler having higher hardness, stress by the cure shrinkage is absorbed, and generation of delamination between the fillers and the piezoelectric elements (the piezoelectric material 110 and the acoustic matching layer 130) can be suppressed. Furthermore, since only either the first grooves 410 or the second grooves 420 divide the uppermost layer (the third acoustic matching layer 130c) of the acoustic matching layer 130, it is possible to reduce an amount of the filler to be charged into the uppermost layer (the third acoustic matching layer 130c) of the acoustic matching layer 130 that is likely to be the start point of delamination Therefore, delamination caused by the cure shrinkage can be reduced. Furthermore, since all of the grooves are not filled with the fillers at a time, it is possible to reduce an amount of the fillers to be charged into the piezoelectric elements (piezoelectric material 110 and the acoustic matching layer 130) at a time. Therefore, influence of the cure shrinkage can be reduced.

Consequently, it is possible to provide the method of manufacturing an ultrasound probe having desired durability, desired acoustic characteristics, and excellent productivity.

### Modified Example

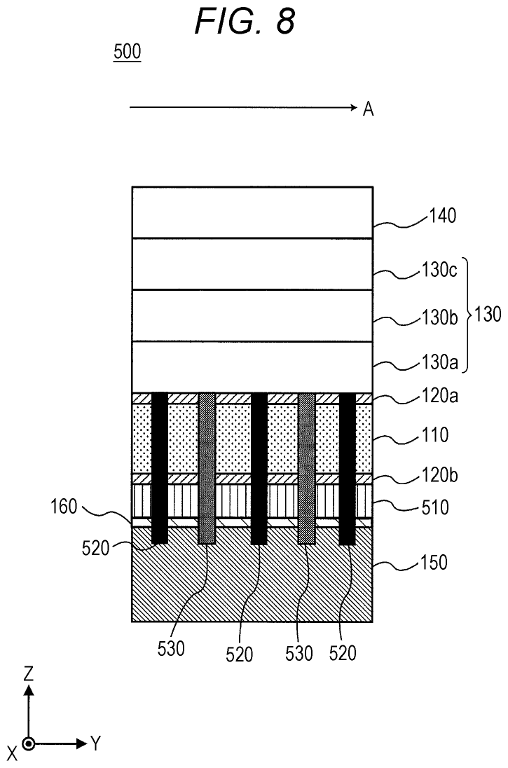

FIG. 8 is a cross-sectional view illustrating an exemplary entire structure of an ultrasound probe according to a modified example of the present invention.

(Configuration of Ultrasound Probe)

In the first to fourth embodiments, the ultrasound probes 100, 200, 300, and 400 each not including a dematching layer have been described. However, an ultrasound probe 500 according to the modified example may include a dematching layer 510. Here, the “dematching layer” refers to a layer that reflects elastic vibration generated by an ultrasound transducer including a piezoelectric element, and also is a layer bonded to the rear surface side of the piezoelectric material 110.

Additionally, the dematching layer 510 is formed from a material having acoustic impedance larger (e.g., 90 MRayls) than acoustic impedance (10 to 30 MRayls) of the piezoelectric material 110, and reflects ultrasound output to an opposite side of a subject (in a direction away from the subject) relative to the piezoelectric material 110.

Materials to be applied to the dematching layer 510 are not particularly limited as far as the materials include tungsten, tungsten carbide, tantalum, or the like. Among these materials, the tungsten carbide is preferable. Alternatively, a tungsten-based alloy obtained by mixing the tungsten carbide with another material such as cobalt may also be applied.

As illustrated in FIG. 8, the ultrasound probe 500 according to the modified example includes the piezoelectric material 110, the signal electrodes 120a and 120b provided to apply the voltage to the piezoelectric material 110, and the acoustic matching layer 130, the acoustic lens 140, the backing material 150, the flexible printed circuit board (FPC) 160, and the dematching layer 510. The ultrasound probe 500 has the signal electrode 120a, the acoustic matching layer 130, and the acoustic lens 140 laminated in this order from the piezoelectric material 110 toward the subject, and has the signal electrode 120b, the dematching layer 510, the flexible printed circuit board (FPC) 160, and the backing material 150 laminated in this order from the piezoelectric material 110 toward an opposite side of the subject.

Here, as illustrated in FIG. 8, the piezoelectric material 110 includes a plurality of first grooves 520 formed substantially in parallel, and second grooves 530 each formed between the plurality of first grooves 520 in a manner substantially in parallel to the first grooves 520, and the piezoelectric material 110 is divided by both the first grooves 520 and the second grooves 530. The number of the first grooves 520 and the number of the second grooves 530 formed in the piezoelectric material 110 may be the same or may be different. Additionally, the dematching layer 510 may be divided by either the first grooves 520 or the second grooves 530. The number of the first grooves 520 and the number of the second grooves 530 formed in the dematching layer 510 may be the same or may be different.

Additionally, either the first grooves 520 or the second grooves 530 that divide the division layer are voids or both kinds of the grooves are respectively filled with fillers having different hardness. As the above-described fillers, as far as the hardness is different, fillers including the same kind of the resin may be used. Here, the fillers to be charged into the first grooves 520 and the second grooves 530 include the resin selected from the group including the epoxy resin, the silicone resin, and the urethane resin described above. For example, the combination of the epoxy resin having the Shore D hardness 80 with the silicone resin having the Shore A hardness 35 is more preferable.

Additionally, as illustrated in FIG. 8, the ultrasound probe 500 according to the modified example may include the backing material 150 arranged on the rear surface side of the piezoelectric material 110. Furthermore, the backing material 150 may include at least either the first grooves 520 or the second grooves 530. In the ultrasound probe 500 according to the modified example, the backing material 150 includes both the first grooves 520 and the second grooves 530.

Additionally, the acoustic matching layer 130 is not divided in the ultrasound probe 500 according to the modified example. In this case, directivity of the ultrasound probe can be more improved by forming the acoustic matching layer 130 from a material having rubber elasticity, such as silicone rubber, chloroprene rubber, ethylene-propylene copolymer rubber, acrylonitrile-butadiene copolymer rubber, and urethane rubber. In this case, the above-described material having the rubber elasticity is preferably a material having a sound speed of 1650 msec or less. However, in the ultrasound probe 500 according to the modified example, the acoustic matching layer 130 may include division layers divided by at least either the first grooves 520 or the second grooves 530 in a manner similar to the first to fourth embodiments.

(Effects)

In the ultrasound probe 500 according to the modified example, either the first grooves 520 or the second grooves 530 are formed as the voids or both kinds of the grooves are respectively filled with the fillers having the different hardness. As a result, cure shrinkage of the fillers can be absorbed even when the respective grooves have the same depths. In particular, since a filler having lower hardness can follow cure shrinkage, even when such a filler is used in combination with a filler having higher hardness, delamination between the fillers and the piezoelectric elements (the piezoelectric material 110 and the acoustic matching layer 130) can be suppressed. In particular, since a filler having lower hardness is more easily deformed and tends to follow the cure shrinkage, when such a filler is used in combination with a filler having higher hardness, stress by the cure shrinkage is absorbed, and generation of delamination between the fillers and the piezoelectric elements (the piezoelectric material 110 and the acoustic matching layer 130) can be suppressed.

Additionally, since the second grooves are formed after the first filler is charged, the stress caused by the cure shrinkage of the first filler can be released. After that, when the second filler is charged, cure shrinkage occurs in a manner similar to the case of the first filler. However, since the stress caused by the cure shrinkage of the first filler is absorbed, generation of delamination caused by the cure shrinkage can be more reduced compared to the case of forming all of the grooves at a time and filling all of the grooves with the fillers at a time. Furthermore, since all of the grooves are not filled with the fillers at a time, it is possible to reduce the amount of the fillers to be charged into the piezoelectric elements (piezoelectric material 110 and the acoustic matching layer 130) at a time. Therefore, the influence of the cure shrinkage can be reduced.

Additionally, the directivity of the ultrasound probe can be more improved by forming the acoustic matching layer from the material having the rubber elasticity, such as the silicone rubber, the chloroprene rubber, the ethylene-propylene copolymer rubber, the acrylonitrile-butadiene copolymer rubber, and the urethane rubber.

Consequently, it is possible to obtain an ultrasound probe having desired durability, desired acoustic characteristics, and excellent productivity.

(Method of Manufacturing Ultrasound Probe)

A method of manufacturing the ultrasound probe 500 according to the modified example can manufacture the ultrasound probe in a manner similar to the flowchart of the first embodiment illustrated in FIG. 2. Accordingly, the steps same as those in the first embodiment are denoted by the same reference signs, and the description thereof will be omitted.

The method of manufacturing the ultrasound probe 500 according to the modified example includes: a first groove forming step (S10) of forming the plurality of first grooves 520 in the piezoelectric material 110; a first filling step (S11) of filling each of the first grooves 520 with the first filler; a second groove forming step (S12) of forming each of the second grooves 530 between the first grooves 520 in the piezoelectric material 110; a second filling step (S13) of filling each of the second grooves 530 with the second filler; a bonding step (S14) of bonding the acoustic matching layer 130 arranged on the subject side of the piezoelectric material 110; and an acoustic lens bonding step (S15) of bonding the acoustic lens 140 to the uppermost layer (the third acoustic matching layer 130c) of the acoustic matching layer 130.

Furthermore, in the method of manufacturing the ultrasound probe 500 according to the modified example, a step of bonding the dematching layer 510 to the rear surface side of the piezoelectric material 110 (the step not illustrated) may be included before the first groove forming step (S10) in addition to the steps illustrated in the flowchart of FIG. 2. Additionally, a step of bonding the backing material 150 to the rear surface side of the dematching layer 510 (the step not illustrated) may also be provided. Furthermore, either the first groove forming step (S10) of forming the plurality of first grooves 520 in the piezoelectric material 110 or the second groove forming step (S12) of forming each of the second grooves 530 between the first grooves 520 in the piezoelectric material 110 may include a step of forming the first grooves 520 or the second grooves 530 in the backing material 150.

Additionally, either the first grooves 520 or the second grooves 530 are the voids or both kinds of the grooves are filled with the fillers respectively having the different hardness. As the above-described fillers, as far as the hardness is different, fillers including the same kind of the resin may be used. Here, the fillers to be charged into the first grooves 520 and the second grooves 530 include the resin selected from the group including the epoxy resin, the silicone resin, and the urethane resin described above. For example, the combination of the epoxy resin having the Shore D hardness 80 with the silicone resin having the Shore A hardness 35 is more preferable. Additionally, as the fillers, as far as the hardness is different, fillers including the same kind of the resin may be used.

(Effects)

In the method of manufacturing the ultrasound probe 500 according to the modified example, either the first grooves 520 or the second grooves 530 are formed as the voids or both kinds of the grooves are respectively filled with the fillers having the different hardness. As a result, cure shrinkage of the fillers can be absorbed. Therefore, it is possible to manufacture an ultrasound probe in which delamination between the fillers and the respective grooves formed in the piezoelectric elements (piezoelectric material 110 and the acoustic matching layer 130) is suppressed. In particular, since a filler having lower hardness is more easily deformed and tends to follow the cure shrinkage, when such a filler is used in combination with a filler having higher hardness, stress by the cure shrinkage is absorbed, and generation of delamination between the fillers and the piezoelectric elements (the piezoelectric material 110 and the acoustic matching layer 130) can be suppressed.

Additionally, since the second groove forming step (S12) is performed after the first filling step (S11), it is possible to release the stress caused by the cure shrinkage of the first filler. After that, when the second filler is charged, cure shrinkage occurs in a manner similar to the case of the first filler. However, since the stress caused by the cure shrinkage of the first filler is absorbed, generation of delamination caused by the cure shrinkage can be more reduced compared to a case of forming all of the grooves at a time and filling all of the grooves with the fillers at a time. Furthermore, since all of the grooves are not filled with the fillers at a time, it is possible to reduce the amount of the fillers to be charged into the piezoelectric elements (piezoelectric material 110 and the acoustic matching layer 130) at a time. Therefore, influence of the cure shrinkage can be reduced.

Moreover, since the first groove forming step (S10) and the first filling step (S11) are performed separately from the second groove forming step (S12) and the second filling step (S13), a desired groove can be filled with a desired filler.

Consequently, it is possible to obtain the ultrasound probe having the desired durability, the desired acoustic characteristics, and the excellent productivity.

(Ultrasound Diagnostic Apparatus)

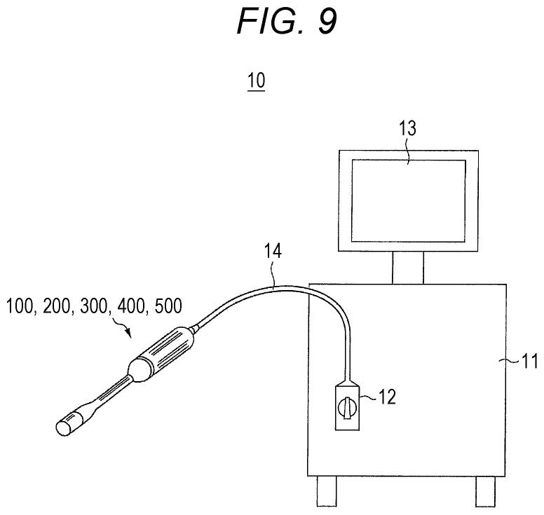

FIG. 9 is a schematic diagram illustrating an exemplary ultrasound diagnostic apparatus 10 including an ultrasound probes 100, 200, 300, 400, or 500. The ultrasound diagnostic apparatus 10 includes the ultrasound probe 100, 200, 300, 400 or 500, a main body 11, a connector 12, and a display 13.

The ultrasound probe 100, 200, 300, 400 or 500 is connected to the ultrasound diagnostic apparatus 10 via a cable 14 connected to the connector 12.

An electric signal (transmission signal) from the ultrasound diagnostic apparatus 10 is transmitted to a piezoelectric material 110 of the ultrasound probe 100, 200, 300, 400, or 500 via the cable 14. This transmission signal is converted into ultrasound by the piezoelectric material 110 and transmitted into a living body. The transmitted ultrasound is reflected at a tissue or the like in the living body, and the reflected wave is partly received by the piezoelectric material 110 again and converted into an electric signal (reception signal), and then transmitted to the main body 11 of the ultrasound diagnostic apparatus 10. The reception signal is converted into image data in the main body 11 of the ultrasound diagnostic apparatus 10 and displayed on the display 13.

The ultrasound diagnostic apparatus in the above-described embodiment can generate an ultrasound image with high image quality because of including the ultrasound probe of the present invention in which an acoustic impedance difference between the piezoelectric material and the subject (living body) is gradually reduced.

Note that the ultrasound probe having the backing material has been described in each of the above-described embodiments, but the ultrasound probe may not necessarily include the backing material. Additionally, an acoustic impedance material equivalent to PZT or greater may be provided between PZT and the backing material, and ultrasound directed to the rear surface side may be reflected so as to overlap with ultrasound directed to the upper surface side.

### Work Examples

In the following, the present invention will be more specifically described with Work Examples, but the present invention is not limited thereto.

Abbreviations used in fabrication of ultrasound probes 1 to 7 are as follows.

Piezoelectric material: Lead zirconate titanate (PZT)

First acoustic matching layer: Cured product of kneaded product obtained by kneading epoxy resin with metal oxide

Second acoustic matching layer: Cured product of kneaded product obtained by kneading epoxy resin with metal oxide

Third acoustic matching layer: Cured product of kneaded product obtained by kneading epoxy resin with rubber particles

(First Filler)

A-1: Two-pack epoxy resin C-1076 having Shore D hardness 80 (manufactured by Tesk Co., Ltd.)

(Second Filler)

B-1: Two-component addition type RTV silicone TSE3032 having Shore A hardness 35 (manufactured by Momentive Performance Materials Holdings Inc.).

B-2: Air

(Charging Methods)

D-1: Method of charging either first filler or second filler at a time

D-2: Method of charging second filler after filling first filler

1-1. Fabrication of Ultrasound Probe 1

First grooves that divide a piezoelectric material were formed in the piezoelectric material at a predetermined interval substantially in parallel (while having a backing material bonded to a rear surface side of the piezoelectric material), and the first filler A-1 was charged and cured at 60° C. for four hours. Next, each of second grooves that divide the piezoelectric material was formed between the first grooves in a manner substantially parallel to the first grooves, and the second filler B-1 was charged and cured at 50° C. for six hours. An acoustic matching layer was bonded, with an adhesive, to an upper surface side of the piezoelectric material filled with the first filler and the second filler, an acoustic lens was further bonded to an uppermost layer of the acoustic matching layer with the adhesive, and thus an ultrasound probe 1 was obtained.

1-2. Fabrication of Ultrasound Probe 2

A first acoustic matching layer and a second acoustic matching layer were bonded to an upper surface side of a piezoelectric material in this order (while having a backing material bonded to a rear surface side of the piezoelectric material). Next, first grooves that divide the piezoelectric material and the acoustic matching layer were formed at a predetermined interval substantially in parallel, and the first filler A-1 was charged and cured at 60° C. for four hours. Next, each of second grooves that divide the piezoelectric material and the acoustic matching layer was formed between the first grooves in a manner substantially parallel to the first grooves, and the second filler B-1 was charged and cured at 50° C. for six hours. Finally, a third acoustic matching layer to be an uppermost layer is bonded to the upper surface side of the second acoustic matching layer, an acoustic lens was further bonded to an upper surface side of the third acoustic matching layer with an adhesive, and thus an ultrasound probe 2 was obtained.

1-3. Fabrication of Ultrasound Probe 3

A first acoustic matching layer and a second acoustic matching layer were bonded to an upper surface side of a piezoelectric material in this order (while having a backing material bonded to a rear surface side of the piezoelectric material). Next, first grooves that divide the piezoelectric material and the acoustic matching layer were formed at a predetermined interval substantially in parallel, and the first filler A-1 was charged and cured at 60° C. for four hours. Next, each of second grooves that divide the piezoelectric material and the acoustic matching layer was formed between the first grooves in a manner substantially parallel to the first grooves, and the second filler B-2 (air) was charged and left as it was. Finally, a third acoustic matching layer to be an uppermost layer was bonded to the upper surface side of the second acoustic matching layer, an acoustic lens was further bonded to an upper surface side of the third acoustic matching layer with an adhesive, and thus an ultrasound probe 3 was obtained.

1-4. Fabrication of Ultrasound Probe 4

First grooves that divide a piezoelectric material were formed in the piezoelectric material at a predetermined interval substantially in parallel (while having a backing material bonded to a rear surface side of the piezoelectric material), and the first filler A-1 was charged and cured at 60° C. for four hours. Next, each of second grooves that do not divide the piezoelectric material was formed between the first grooves in a manner substantially in parallel to the first grooves, and the second filler B-1 was charged and cured at 50° C. for six hours. An acoustic matching layer was bonded with an adhesive to an upper surface side of the piezoelectric material filled with the first filler and the second filler, an acoustic lens was further bonded to an uppermost layer of the acoustic matching layer with the adhesive, and thus an ultrasound probe 4 was obtained.

1-5. Fabrication of Ultrasound Probe 5

A first acoustic matching layer and a second acoustic matching layer were bonded to an upper surface side of a piezoelectric material in this order (while having a backing material bonded to a rear surface side of the piezoelectric material). Next, first grooves that divide the piezoelectric material and the acoustic matching layer were formed at a predetermined interval substantially in parallel, and the first filler A-1 was charged and cured at 60° C. for four hours. Next, a third acoustic matching layer to be an uppermost layer was bonded to an upper surface side of the second acoustic matching layer. Each of second grooves that divide the piezoelectric material and the acoustic matching layer was formed between the first grooves in a manner substantially parallel to the first grooves in the piezoelectric material having the third acoustic matching layer bonded, and the second filler B-1 was charged and cured at 50° C. for six hours. Finally, an acoustic lens was bonded to an upper surface side of the third acoustic matching layer with an adhesive, and thus an ultrasound probe 5 was obtained.

1-6. Fabrication of Ultrasound Probe 6

A first acoustic matching layer and a second acoustic matching layer were bonded to an upper surface side of a piezoelectric material in this order (while having a backing material bonded to a rear surface side of the piezoelectric material). Next, first grooves that divide the piezoelectric material and the acoustic matching layer were formed at a predetermined interval substantially in parallel, and the first filler A-1 was charged and cured at 60° C. for four hours. Next, a third acoustic matching layer to be an uppermost layer was bonded to an upper surface side of the second acoustic matching layer. Each of second grooves that divide the piezoelectric material and the acoustic matching layer was formed between the first grooves in a manner substantially in parallel to the first grooves in the piezoelectric material having the third acoustic matching layer bonded, and the second filler B-2 (air) was filled and left as it was. Finally, an acoustic lens was bonded to an upper surface side of the third acoustic matching layer with an adhesive, and thus an ultrasound probe 6 was obtained.

1-7. Fabrication of Ultrasound Probe 7

A first acoustic matching layer, a second acoustic matching layer, and a third acoustic matching layer were bonded to an upper surface side of a piezoelectric material in this order (while having a backing material bonded to a rear surface side of the piezoelectric material). Next, grooves that divide the piezoelectric material and the acoustic matching layers were formed at predetermined intervals substantially in parallel, and the filler B-1 was charged and cured at 50° C. for six hours. Finally, an acoustic lens was bonded to an upper surface side of the third acoustic matching layer with an adhesive, and thus an ultrasound probe 7 was obtained.

Table 1 shows specifications of the ultrasound probes 1 to 7.

TABLE 1

| | First filler | Second filler | Charging method |
|---|---|---|---|
| 1 | A-1 | B-1 | D-2 |
| 2 | A-1 | B-1 | D-2 |
| 3 | A-1 | B-2 | D-2 |
| 4 | A-1 | B-1 | D-2 |
| 5 | A-1 | B-1 | D-2 |
| 6 | A-1 | B-2 | D-2 |
| 7 | B-1 | B-1 | D-1 |

2. Evaluation

Generation of delamination was evaluated by using the fabricated ultrasound probes 1 to 7. The results are shown in Table 2.

(Evaluation Method)

Each of the ultrasound probes 1 to 7 was cut by a dicing saw (manufactured by Disco Corporation) into a portion having a thickness of 5×5 mm, and each cut surface thereof was observed by using a scanning electron microscope (manufactured by Hitachi High-Tech Corporation).

(Evaluation Criteria)

∘: No delamination or delamination of less than 1 μm was observed between the acoustic matching layer, the piezoelectric material, the flexible printed circuit board, or the backing material and any one of the fillers

x: Delamination of 1 μm or more was observed between the acoustic matching layer, the piezoelectric material, the flexible printed circuit board, or the backing material and any one of the fillers

TABLE 2

| | 1 | 2 | 3 | 4 | 5 | 6 | 7 |
|---|---|---|---|---|---|---|---|
| Generation of | ∘ | ∘ | ∘ | ∘ | ∘ | ∘ | x |
| delamination | | | | | | | |

In the ultrasound probes, either the first grooves or the second grooves were formed as voids or both kinds of the grooves were respectively filled with the fillers having the different hardness. Therefore, it was found that cure shrinkage of the fillers could be absorbed and it was possible to hardly cause delamination between the piezoelectric elements (piezoelectric material and the acoustic matching layer) and any one of the fillers during manufacture. Since a filler having lower hardness is more easily deformed and tends to follow cure shrinkage, it can be considered that: when such a filler is used in combination with a filler having higher hardness, stress by the cure shrinkage can be absorbed, and therefore, generation of delamination between each of the fillers and the piezoelectric elements (the piezoelectric material and the acoustic matching layer) could be suppressed.

Additionally, it was found that: compared to the case where all of the grooves were filled with the fillers at a time, generation of delamination caused by cure shrinkage could be more reduced by forming the second grooves and charging the second filler after charging the first filler. The reason may be that: since all of the grooves were not filled with the fillers at a time, an amount of the fillers to be charged into the piezoelectric elements (piezoelectric material and the acoustic matching layer) at a time is reduced, and therefore, influence of the cure shrinkage is hardly received. Additionally, another reason may be that: the stress caused by the cure shrinkage of the first filler could be released by forming the second grooves and charging the second filler after charging the first filler.

The present invention is applicable as an ultrasound probe of an ultrasound device intended to obtain an ultrasound image with excellent sensitivity and high image quality.

Although embodiments of the present invention have been described and illustrated in detail, the disclosed embodiments are made for purposes of illustration and example only and not limitation. The scope of the present invention should be interpreted by terms of the appended claims.

What is claimed is:

1. An ultrasound probe comprising:
a piezoelectric material in which piezoelectric elements to transmit and receive ultrasound are one-dimensionally arrayed; and
at least one acoustic matching layer arranged on a subject side of the piezoelectric material,
wherein the piezoelectric material includes a plurality of first grooves, and at least a second groove formed between the plurality of first grooves,
the piezoelectric material is divided by at least either the first grooves or the second groove, and
either each of the first grooves or the second groove is a void, or the first grooves and the second groove are respectively filled with fillers having different hardness.

2. The ultrasound probe according to claim 1, wherein the acoustic matching layer includes a division layer divided by at least either the first grooves or the second groove.

3. The ultrasound probe according to claim 2, wherein the division layer is a layer divided by only either the first grooves or the second groove.

4. The ultrasound probe according to claim 2, wherein the division layer is an uppermost layer of the acoustic matching layer.

5. The ultrasound probe according to claim 1, wherein the piezoelectric material is divided by only either the first grooves or the second groove.

6. The ultrasound probe according to claim 1, further comprising a backing material arranged on a rear surface side of the piezoelectric material,
wherein the backing material includes at least either the first grooves or the second groove.

7. The ultrasound probe according to claim 1, wherein the filler includes a resin selected from a group including an epoxy resin, a silicone resin, and a urethane resin.

8. An ultrasound probe comprising:
a piezoelectric material in which piezoelectric elements to transmit and receive ultrasound are one-dimensionally arrayed; and
at least one acoustic matching layer arranged on a subject side of the piezoelectric material,
wherein the piezoelectric material includes a plurality of first grooves and a second groove formed between the plurality of first grooves, and
the acoustic matching layer includes a division layer divided by only either the first grooves or the second groove.

9. A method of manufacturing an ultrasound probe including a piezoelectric material in which piezoelectric elements are one-dimensionally arrayed, the method comprising:
forming a plurality of first grooves in the piezoelectric material;
filling the plurality of first grooves with a first filler;
forming a second groove between the plurality of first grooves in the piezoelectric material;
filling the second groove with a second filler; and
bonding at least once an acoustic matching layer arranged on a subject side of the piezoelectric material,
wherein at least either forming the first grooves or forming the second groove includes forming a groove that divides the piezoelectric material, and
either the first filler or the second filler is air, or the first filler and the second filler are fillers respectively having different hardness.

10. The method of manufacturing an ultrasound probe according to claim 9, wherein the bonding is performed before forming the second groove, and includes bonding a division layer divided by the second groove.

11. The method of manufacturing an ultrasound probe according to claim 10, wherein bonding the division layer is performed between filling the first filler and forming the second groove.

12. The method of manufacturing an ultrasound probe according to claim 10, wherein the division layer is an uppermost layer of the acoustic matching layer.

13. The method of manufacturing an ultrasound probe according to claim 9, further comprising bonding the piezoelectric material to a backing material,
wherein at least either forming the first grooves or forming the second groove includes forming the first grooves or the second groove in the backing material.

14. The method of manufacturing an ultrasound probe according to claim 9, wherein the first filler includes a resin selected from a group including an epoxy resin, a silicone resin, and a urethane resin.

15. The method of manufacturing an ultrasound probe according to claim 9, wherein the second filler includes a resin selected from a group including an epoxy resin, a silicone resin, and a urethane resin.

16. A method of manufacturing an ultrasound probe including a piezoelectric material in which piezoelectric elements are one-dimensionally arrayed, the method sequentially comprising:
forming a plurality of first grooves in the piezoelectric material;
bonding at least once an acoustic matching layer arranged on a subject side of the piezoelectric material; and
forming a second groove between the plurality of first grooves in the piezoelectric material having the acoustic matching layer bonded.

17. An ultrasound diagnostic apparatus comprising the ultrasound probe according to claim 1.

18. An ultrasound diagnostic apparatus comprising the ultrasound probe according to claim 8.
